(19) United States
(12) Plant Patent Application Publication
(10) Pub. No.: US 2003/0066115 P1
(43) Pub. Date: Apr. 3, 2003

(54) Unique variety of geranium

(76) Inventor: Charles F. Heidgen, Batavia, IL (US)

Correspondence Address: Charles F. Heidgen, 821 Walnut Street, Batavia, IL, 60510, US

(21) Appl. No.: 09/969,331

(22) Filed: Oct. 3, 2001

(51) Int. Cl. A01H 5/00

(52) U.S. Cl. PLT/325

(57) ABSTRACT

A geranium plant named ‘Mrs. Peters’, from a chimeral sport of Hills of Snow identified by its medium to vigorous growth habit; its medium-sized, grey green foliage with irregular, creamy white edges; and its prolific flowering characteristic. The blooms are medium sized, double flowers that are a distinct true pink color that gradually fades to white at the center of each of the blooms.

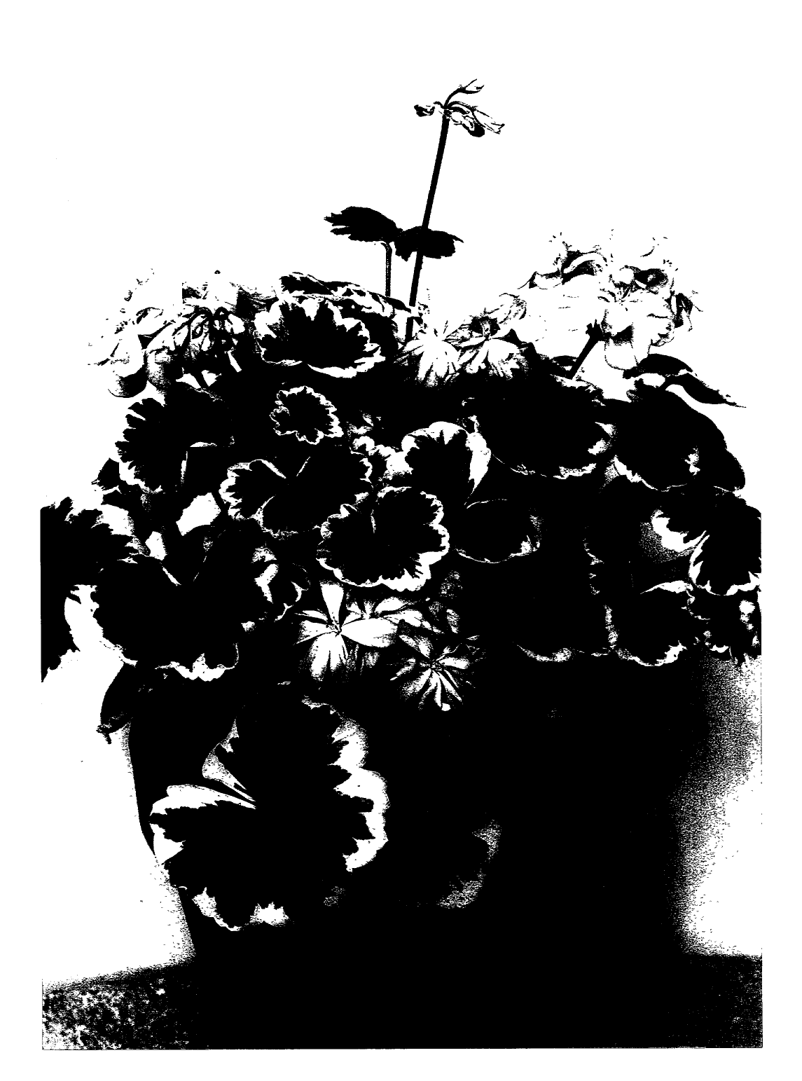

### BACKGROUND OF THE INVENTION

The present invention, hereinafter referred to by the cultivar name “Mrs. Peters” relates to a new and distinct geranium. “Mrs. Peters” is a new and improved plant resulting from a planned breeding program as performed by me in my greenhouse at 821 Walnut Street, Batavia, Ill. My breeding program began in March 1977, and has been ongoing since that time. This plant was originally discovered in July of 1998 as a chimeral sport of Hills of Snow, which is a green and white variegated foliaged geranium variety that has a narrow border of white on silvery gray green leaves. Hills of Snow is a very old, non-patented variety of Fancy Leaf Zonal Geranium. The botanical classification Pelargonium×hortorum would be appropriate for this plant.

Upon recognition of the outstanding characteristics of this plant, it was selected from the progeny of the stated cross, and was set aside for further observation and testing and was subsequently named “Mrs. Peters.” Asexual reproduction of “Mrs. Peters” has been repeatedly accomplished by the rooting of vegetative cuttings in my greenhouse in Batavia, Ill. Examination of the plants so propagacea through a series of propagations has demonstrated that the combination of characteristics herein disclosed for “Mrs. Peters” are firmly fixed and retained through successive generations through asexual reproduction.

### SUMMARY OF THE INVENTION

The new and distinguishing characteristics of “Mrs. Peters” reside primarily in its medium to vigorous growth habit, its prolific blooming characteristic and its medium-sized, grey green foliage with creamy white edges. This geranium also has medium sized, double flowers with a true pink color that fades to white at the center, as well as heat tolerance. This plant might be seen as a novel and desirable introduction within the industry by a combination of desired traits including:

Being particularly adapted to culture in the ground or in pots due to its medium to vigorous growth habit, dense leafy canopy and its tendency to branch prolifically. Forming a bushy plant mass in approximately 7-9 weeks from one plantlet, and a plant that is not stretched or leggy, but rather, a mounded and dense plant.

Having a generous amount of foliage, with grey green coloration and contrasting, creamy white variegated edges on each of the medium sized leaves and of higher than normal density.

Presenting a high number of well filled umbels of varied states of maturity placed well above the foliage mass, and having a true pink floret coloration, that fades to white at the center, which is distinctively, contrastingly highlighted over and against the grey green, variegated foliage.

Having a large number of umbels with a generous number of open, double petal florets of a medium size and appearance, borne on pedicels which present the florets in a dense arrangement to form umbels having a globular and somewhat fluffy appearing shape.

Presenting a large number of umbels which also vary in stage of maturity, which gives the plant an attractive appearance for an extended period of time, rather than being one that blooms in discontinuous flushes.

The color definitions to follow have been taken from The Royal Horticultural Colour Chart of The Royal Horticultural Society, London, where presented by the letters RHS followed by a color grouping indication, the number indicating the color patch and the letter indicating the shade, except where color definitions in general terms of ordinary meaning are presented. Plant descriptions were made from specimens grown under greenhouse conditions during daylight hours in Batavia, Ill.

### BRIEF DESCRIPTION OF THE DRAWING

The photograph depicts a market unit that has one market stage specimen of the plant growing in a 5½″ pot, in a stage of inflorescence. The assemblage is shown in a side view, depicting the characteristics of branching, leaves, leaf coloration and zonation, the number and varied stages of buds and flowers, and the positioning of the flower heads relative to the mass of the plants in the pot.

### BOTANICAL DESCRIPTION OF THE PLANT

Botanical classification: Pelargonium×hortorum

Market class: Zonal Geranium suitable for planting in the ground or in containers.

Market name: “Mrs. Peters”.

Growth: “Mrs. Peters” is a prolific blooming Geranium with double flowers and the truss or flower head is of medium size. The growth is upright in habit and given to branching freely.

Growth rate: Average, producing a typical 4½″ flowering geranium in about 7 to 9 weeks from one 2¼″ plantlet using standard commercial culture.

Blooming habit: Exceptionally profuse and continuously blooming plant throughout the year, outdoors during the frost-free period, or in the greenhouse; a mature 4½″ pot commonly producing a range of 4 to 7 flower heads at one time, in varying states of maturity.

### INFLORESCENCE

Flower heads:

Size.—2⅝ to 3¼ inches across when the florets are fully open.

Count.—3 to 6 present at one time.

Truss:

Size.—Medium to large size; ranges from 2⅝ to 3¼ inches in diameter.

Shape.—Rather dense, a somewhat fluffy appearing globe, as it opens.

Number of florets.—Variable, range from approximately 13 to 29, average 14 to 20.

Lasting quality.—Average. Blooms last 5 to 8 days, possibly longer in cooler, dry weather.

Buds:

Sepals.—Shape- Long, average width, pointed. Color- RHS Green Group, 143 C, with RHS White Group 155 C edges.

Color.—RHS Yellow Green Group, 145 A, with RHS White Group 157 B edges.

Form.—Short to medium length, slight point, but more rounded.

Florets:

Number.—14 to 20.

Form.—Rather cupped to flat, as it opens fully.

Size.—Ranging from ⅞ inch to 1⅝ inches across a fully open floret.

Petals:

Number.—Ranging from 12 to 17.

Size.—Ranging from about 7/16 to ⅞ inches long and approximately ⅜ to 9/16 inches across.

Texture.—Soft.

Appearance.—Bright.

Color.—RHS Red Group, 55 B main part and toward edge of the petal with a RHS White Group 155 D at the center.

Petaloids:

Number.—Substantially absent, but may occur in number up to 8.

Shape.—Variable, often misshapen.

Color.—Very similar to that of the petals.

Size.—3/16 to ⅜ inches long and ¼ to ⅝ inches long.

Sepals:

Number.—Usually 5.

Color.—RHS Green Group, 143 C with RHS White Group 155 C edges.

Pedicel:

Length.—About ⅞ to ⅜ inches long.

Color.—RHS Yellow Green Group, 146 C.

Peduncle:

Arises.—From node, opposed to leaf petiole.

Pubescence.—Normal.

Color.—RHS Yellow Green Group, 144 A.

### REPRODUCTIVE ORGANS

Androecium:

Stamens.—Number- 4 to 8. Shape- Ribbon-like filaments jointed at bases. Color- Pale pink. Anther Attachment versatile.

Anthers.—Number- 4 to 8. Shape- Normal, well developed. Color- Brown.

Gynoecium:

Pistil.—Number- One, compound. Length- Within the range of 3/32 to 5/32 inches.

Stigma.—Color- Rose pink.

Style.—Color- Pink fading to white toward ovary.

Ovary.—Size- Approximately 1/32 to 1/16 inches long.

Fertility: Normal, viable seed is rarely produced.

Behavior: Once the plant begins to bloom, it has a prolific blooming habit and yields a striking display of medium sized, double flowers of true pink color that fades to white at the center of each floret against its medium sized, grey green leaves with creamy white, variegated edges.

General observations: Inflorescence is of medium size and of rather dense heads of double florets, which are an attractive contrast to the grey green foliage with creamy white variegated edges, approximately 3 inches in diameter. At optimum stage of opening, the double florets are distinctive and striking with their true pink coloration that fades to white at the center. Flowers have high heat tolerance when compared to other varieties.

### STEM

Thickness: Medium to thick.

Internodes: Medium to short.

Branching: Very free breaking.

Attitude: Upright, bushy habit.

### FOLIAGE

Leaves:

Quantity.—Abundant.

Shape.—Reniform.

Size.—Medium, largest leaves range from about 2⅝ to 3⅝ inches across.

Margin.—Crenate.

Texture.—Somewhat pubescent, matt texture.

Coloration.—RHS Greyed Green Group, 191 A, with an irregularly shaped variegation of RHS White Group 155 A on the edge of each leaf. The variegation ranges in width from 1/16 to ⅞, beginning at the edge of the leaf and going toward the center of each leaf.

Petioles:

Texture.—Lightly pubescent.

Length.—Approximately 1⅜ to 2⅛ inches.

Color.—RHS Yellow Green Group, 146 B.

“Mrs. Peters” is comparable to “Wilhelm Languth” with which it shares market class, but is easily distinguishable therefrom in terms of flower color; “Mrs. Peters” having true pink petal coloration which fades to white at the center of each floret, while the petals of “Wilhelm Languth” are scarlet red with no gradation of color and no white eye. “Mrs. Peters” is readily distinguished from “Peaches” by having double florets of true pink petal coloration fading to a white center, in addition to a grey green leaf coloration with a bold, creamy white, irregular variegation on the edges of each leaf.

I claim:

1. A new and distinct variety of Geranium plant, substantially as shown and described, characterized by its upright, bushy growth habit; its medium-sized grey green foliage with creamy white, irregular variegation on the edges of each leaf; its true pink double flowers that fade to white at the center; its abundant blooming habit and its heat tolerance.
